(19) United States
(12) Patent Application Publication
(10) Pub. No.: US 2011/0306600 A1
(43) Pub. Date: Dec. 15, 2011

(54) NOVEL THERAPEUTIC COMPOUNDS

(75) Inventor: Ramesh SESHA, Windsor, NJ (US)

(73) Assignee: Grunenthal GmbH, Aachen (DE)

(21) Appl. No.: 13/157,347

(22) Filed: Jun. 10, 2011

Related U.S. Application Data
(60) Provisional application No. 61/397,401, filed on Jun. 11, 2010.

(51) Int. Cl. A61K 31/16; C07C 237/24; C07D 333/24; C07C 237/20; C07D 265/30; C07C 271/64; C07D 491/06; C07D 211/42; C07C 229/34; C07C 271/54; A61K 31/381; A61K 31/5375; A61K 31/165; A61K 31/27; A61K 31/55; A61K 31/4418; A61K 31/216; A61K 31/225; A61P 25/00; A61P 25/24; A61P 25/28; A61P 25/16; A61P 25/30; A61P 29/00; A61P 1/00; A61P 35/00; A61P 25/04; C07C 237/14

(52) U.S. Cl. 514/215; 564/193; 564/191; 549/77; 564/196; 544/162; 564/163; 560/159; 560/125; 540/581; 546/300; 560/173; 560/171; 560/160; 560/170; 514/626; 514/613; 514/438; 514/237.5; 514/616; 514/490; 514/484; 514/351; 514/529; 514/551; 560/115; 514/548

(57) ABSTRACT

The invention discloses a number of therapeutic compounds and a method of treating a disorder in mammal.

### FIELD OF INVENTION

The invention describes number of novel therapeutically active compounds that are useful for treating a disorder in a mammal.

### BACKGROUND OF THE INVENTION

The invention had the object of finding novel compounds having valuable properties, in particular those which can be used for the preparation of medicaments.

The compounds of this can be used to treat a number of disorders and they include neurodegenerative disorders, including central nervous system disorders such as depression, Alzheimer's disease, cognitive disorders, motor disorders such as Parkinson's disease, drug addiction, behavioral disorders and inflammatory disorders, stomach disorders, cancers, and also serve as analgesics in the treatment of acute, chronic or recurrent pain.

There are a number of classes of therapeutic compounds used for treating various disorders including pain. These include acetaminophen, NSAIDs such as naproxen, meloxicam etc, Selective serotonin reuptake inhibitor (SSRIs) like citalopram and fluoxetine etc, CINODS such as naproxcinod, OPIATES such as morphine, tramadol, tapentadol, oxycodone etc, Antiepileptic drugs such as gabapentin, pregabalin, NMDA (N-methyl-D-aspartic acid) Receptor antagonists like memantine, Norepinephrine Reuptake inhibitors such as reboxetine and atomoxetine, and Serotonin Norepinephrine Reuptake inhibitors (SNRIs) such as duloxetine etc and Acetylcholinestearase inhibitors such as rivastigmine and donepezil etc.

Opioid or opioid agonists class of drugs include morphine, the archetypical opioid, and various others such as, for example, codeine, dihydrocodeine, hydrocodone, hydromorphone, levorphanol, meperidine, buprenorphine, fentanyl, fentanyl derivatives, dipipanone, heroin, tapentadol tramadol, etorphine, dihydroetorphine, butorphanol, methadone, diamorphine, oxycodone, oxymorphone, pethidine and propoxyphene, etc. Opioid agonists chemically interact with areas or binding sites of the central nervous system related to the perception of pain, to movement, mood and behavior, and to the regulation of neuroendocrinological functions. Opioid agonists exhibit pharmacological properties that provide a range of therapeutic uses for patients in addition to analgesic use. Opioid agonists have been prescribed for effective use as hypnotics, sedatives, anti-diarrheal, anti-spasmodic, and anti-tussives.

Similarly, GABA (g-aminobutyric acid) is one of the major inhibitory transmitters in the central nervous system of mammals. A number of antiepileptic compounds such as GABA analogues (also referred to as voltage gated calcium channel blockers or alpha 2-delta ligands) with considerable pharmaceutical activity have been synthesized in the art. GABA analogue cis-4-aminocrotonic acid (3) selectively activates a third class of GABA receptors in the mammalian CNS.

Serotonin Norepinephrine Reuptake inhibitors (SNRIs) are a class of antidepressant used in the treatment of clinical depression and other affective disorders. They are also sometimes used to treat anxiety disorders, obsessive-compulsive disorder, attention deficit hyperactivity disorder (ADHD) and chronic neuropathic pain. They act upon two neurotransmitters in the brain that are known to play an important part in mood, namely, serotonin and norepinephrine. Examples of SNRIs include Venlafaxine, duloxetine, milnacipran and desvenlafaxine etc.

Another class of therapeutic targets includes NMDA (N-methyl-D-aspartic acid) Receptors. The NMDA antagonists inhibit the action of the N-methyl d-aspartate receptor (NMDAR) and they include ketamine (K), dextromethorphan (DXM), phencyclidine (PCP) often used as analgesic agents.

Acetylcholinesterase is one of the most crucial enzymes for nerve response and function. Acetylcholinesterase (AChE) catalyzes the hydrolysis of acylcholinesters with a relative specificity for acetylcholine and is used for developing drugs to treat neurodegerative disorders.

Norepinephrine Reuptake inhibitors (NRI, NERI) or adrenergic reuptake inhibitor (ARI), is a type of drug which acts as a reuptake inhibitor for the neurotransmitters norepinephrine (noradrenaline) and epinephrine (adrenaline) by blocking the action of the norepinephrine transporter. They include Atomoxetine/Tomoxetine (Strattera), Mazindol (Mazanor, Sanorex), Reboxetine (Edronax, Vestra) and Viloxazine (Vivalan).

However, all these classes of drugs have one or more side effects that limit their use in treating a disorder for example- particularly over a long period of time. There is an unmet need to invent and develop better and novel chemical compounds that target one or more of this class of therapeutic targets.

The present inventor while working on novel therapeutic molecules invented a number of compounds that provide therapeutic benefits.

### SUMMARY OF THE INVENTION

The present invention provides novel compounds that have been found to possess therapeutic activity. Accordingly, compounds of the invention are expected to be useful and advantageous as therapeutic agents for those diseases and disorders that can be treated by modulation of number receptors including but not limited to opiate, acetylcholinsterase, NMDA receptors, serotonin and/or norepinephrine reuptake.

One aspect of the invention relates to compounds of formula 1-40:

The present invention also relates to pharmaceutical compositions comprising the compounds of the present invention and a pharmaceutically acceptable carrier.

The present invention also relates to methods for the treatment or prevention of disorders, diseases, or conditions in a mammal in need thereof by administering the compounds and pharmaceutical compositions of the present invention.

The present invention further relates to the use of the compounds of the present invention in the preparation of a medicament useful for the treatment or prevention of disorders, diseases, or conditions in a mammal in need thereof by administering the compounds and pharmaceutical compositions of the present invention.

Another aspect of the invention relates to a method of treating conditions comprising the step of administering a compound of formula (1-35); wherein said condition is selected from the group consisting of psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Another aspect of the invention relates to a method of treating conditions comprising the step of administering a compound of formula (III); wherein said condition is selected from the group consisting of schizophrenia, bipolar disorder, and obsessive-compulsive disorder.

### BRIEF DESCRIPTION OF FIGURES

FIG. 1 is a graph showing the time course of antinociceptive effect of compound of formula 2 against placebo in the thermal tail flick test.

FIG. 2-5 are schematic diagrams of representative methods of manufacturing the compounds of this invention.

### DETAILED DESCRIPTION OF THE INVENTION

The term “dosage form” as used herein is defined to mean a solid oral pharmaceutical preparation or system in which doses of medicine or active drug are included. A dosage form will desirably comprise, for example, at least one slow release dosage form including various slow release forms such as, osmosis controlled-release dosage form, erosion controlled-release dosage form, dissolution controlled-release dosage form, diffusion controlled-release dosage form, controlled-release matrix core, controlled-release matrix core coated with at least one release-slowing coat, enteric coated dosage form, one sustained dosage, dosage form surrounded by at least one delayed-release coat, capsules, minitablets, caplets, uncoated microparticles, microparticles coated with release-slowing coat, microparticles coated with delayed-release coat or any combination thereof.

The term “effective amount” as used herein means a dosage which is sufficient in order for the treatment of the patient to be effective compared with no treatment.

The term “medicament” as used herein means a dosage form suitable for administration of the pharmaceutically active compound to a patient.

The term “optimal” us used herein means an amount which is the optimal dosage for that compound when used in single-compound therapy.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “pain and pain related conditions” as used herein is defined as any pain due to a medical conditions including but not limited to neuropathic pain, osteoarthritis, rheumatoid arthritis, fibromyalgia, and back, musculoskeletal pain, Ankylosing spondylitis, juvenile rheumatoid arthritis, migraines, dental pain, abdominal pains, ischemic pain, postoperative pain or because of an anesthetic or surgical contrition.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “therapeutically effective amount” means an amount that elicits a biological response in a mammal including the suboptimal amount.

The term “Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

The term “Saturated, partially-saturated or unsaturated” includes substituents saturated with hydrogens, substituents completely unsaturated with hydrogens and substituents partially saturated with hydrogens.

The term “Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.

The term “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

The term “Silyl protecting groups” are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino group provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

The compounds of this invention have been prepared using a number starting materials outlined in Formula I to XII.

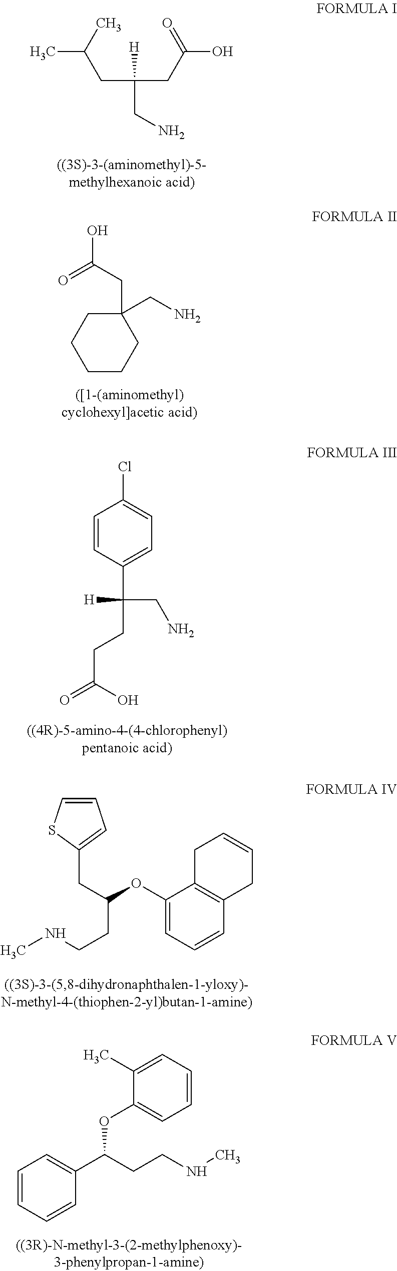

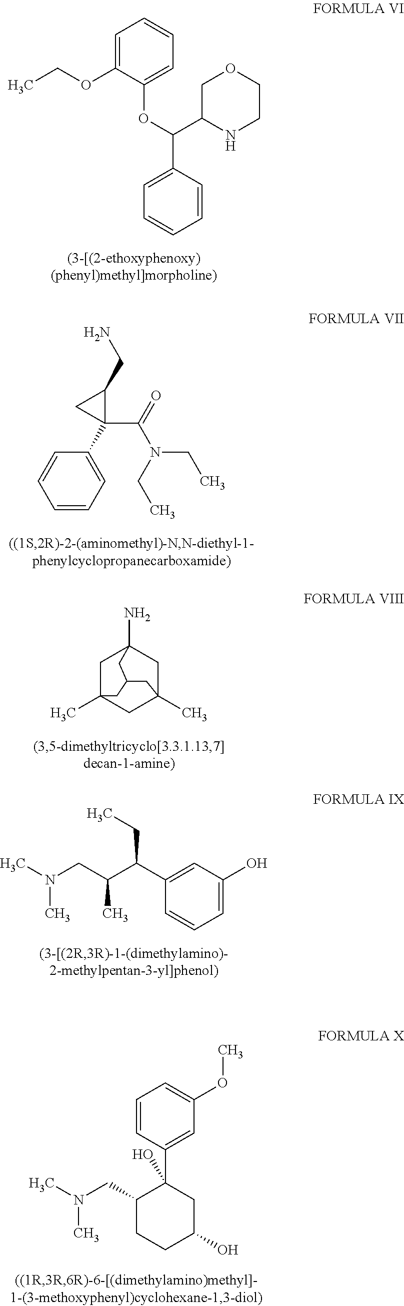

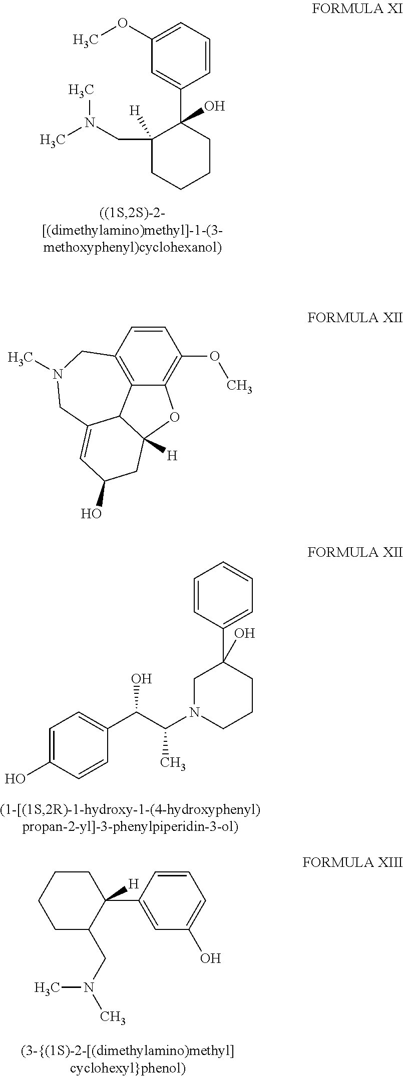

The starting materials were converted a variety of reactive starting materials such acid chlorides or protected starting material according to standard procedures known to the skilled in the art. Many procedures are available for forming amide bonds between an amine derivative formula (IV to IX) and a carboxylic acid chloride (I to III) with the use of coupling agents. For Example the Amide formation was carried out according to the Scheme I. Procedures have been developed which use reagents such as carbodiimides as amide coupling agents. These carbodiimides include for example dicyclohexylcarbodiimide (DCC), diisopropylcarbodiimide (DIC), 1-(3-dimethylaminopropyl)-3-ethylcarbodiimide hydrochloride (EDC) and the like. Other amide coupling agents known in the art such as 1-ethoxycarbonyl-2-dihydroquinoline (EEDQ), phosphonium (e.g. phosphonium hexafluorophosphate (BOP), and others) or uronium-based reagents (e.g. TBTU, HATU and others) may also be used to form the amide bonds. In addition, anhydrides may also be utilized to form the desired amide bond. Catalysts such as 1-hydroxybenzotriazole (HOBT) and derivatives thereof have also been used. A summary of such methods is found in “Comprehensive Organic Transformations”, R. C. Larock, VCH Publishers (1989) pp. 972-972. An overview of such transformations is also available in “March's Advanced Organic Chemistry: Reactions, Mechanisms, and Structure (Sixth Edition)”, Michael B. Smith and Jerry March, Wiley-Interscience Publishers, (2007), pp 1431-1434.

Another general reaction for the preparation of amides is the treatment of acyl halides with amine. Such a transformation is well known to those skilled in the art and an overview of such transformations is available in “March's Advanced Organic Chemistry: Reactions, Mechanisms, and Structure (Sixth Edition)”, Michael B. Smith and Jerry March, Wiley-Interscience Publishers, (2007), pp. 1427-1429.

Esterification was another mode of preparing the novel chemical entities as per this invention. A typical preparation was prepared according to Scheme II.

Similarly the cabamate compounds (Example X-X) were prepared using an efficient solvent-free methodology in high yield and purity from relevant starting compounds, sodium cyanate and silica sulfuric acid (Scheme X). The typical Carbamate reaction was carried according to the Scheme III.

In all these reactions, protecting groups were employed to protected desired amino or carboxyl groups. The method of protecting desired groups are known to persons skilled in the art.

A number of protecting groups are used to protect amine or carboxyl group that needs to be retained in the final compound. Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

The invention further contemplates separating the enantioners in whole or in part of the present invention or synthesizing enantiomerically enriched compounds of the invention. The composition may be prepared by separating the enantioners in whole or in part by standard methods, for example by chemical resolution using optically active acid or by use of column chromatography or reverse-phase column chromatography using a substantially optically active (or “chiral”) stationary phase as known to those skilled in the art. The formation and/or isolation of specific enantiomers of a compound is not routine, and there are no general methods that may be used to obtain specific enantiomers of all compounds. The methods and conditions used to obtain specific enantiomers of a compound must be determined for each specific compound.

Enantiomerically enriched compounds of the invention can also be obtained from enantiomerically enriched precursors.

One aspect of the invention relates to compounds of formula 1:

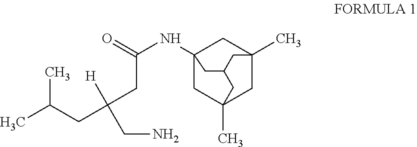

Another aspect of the invention relates to compounds of formula 2:

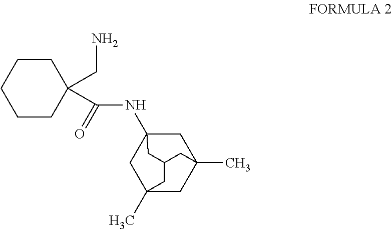

Another aspect of the invention relates to compounds of formula 3;

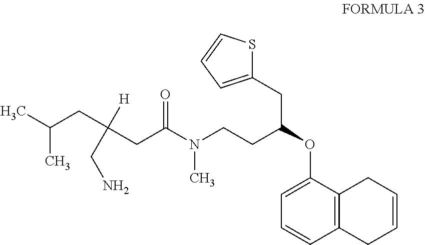

Another aspect of the invention relates to compounds of formula 4;

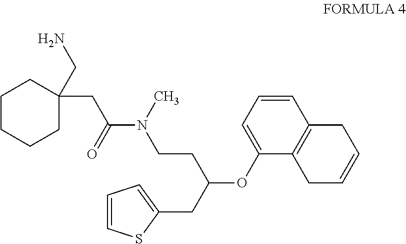

Another aspect of the invention relates to compounds of formula 5;

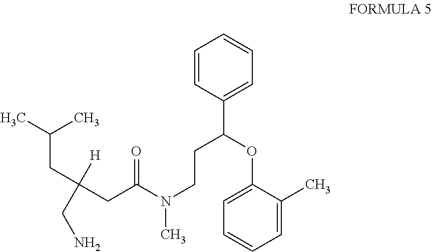

Another aspect of the invention relates to compounds of formula 6;

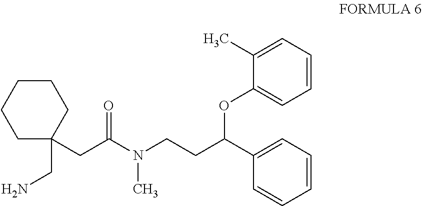

Another aspect of the invention relates to compounds of formula 7;

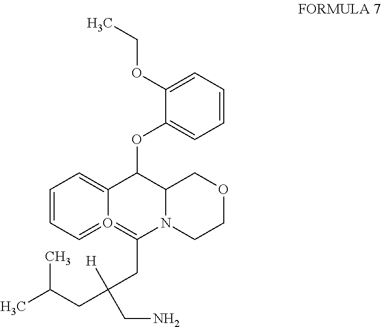

Another aspect of the invention relates to compounds of formula 8;

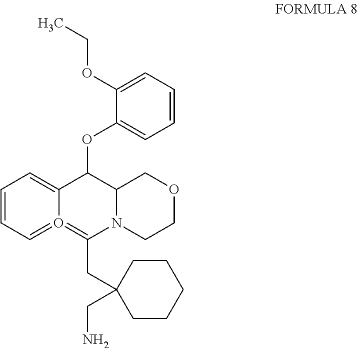

Another aspect of the invention relates to compounds of formula 9;

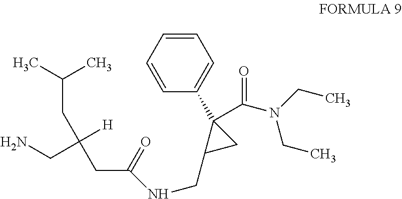

Another aspect of the invention relates to compounds of formula 10;

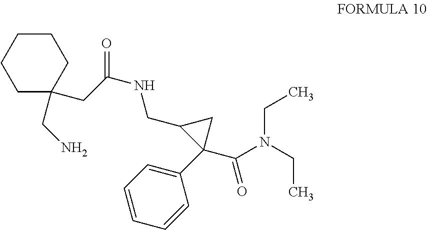

Another aspect of the invention relates to compounds of formula 11;

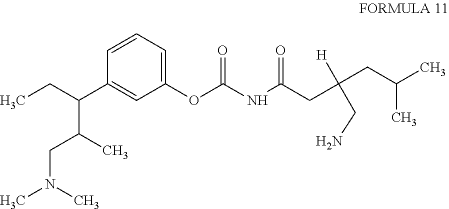

Another aspect of the invention relates to compounds of formula 12;

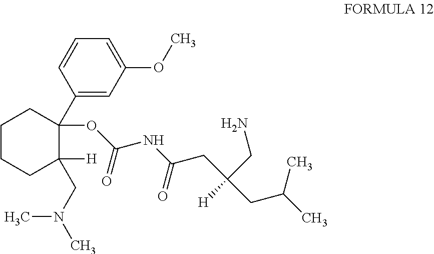

Another aspect of the invention relates to compounds of formula 13;

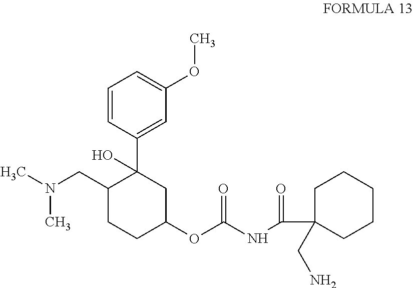

Another aspect of the invention relates to compounds of formula 14;

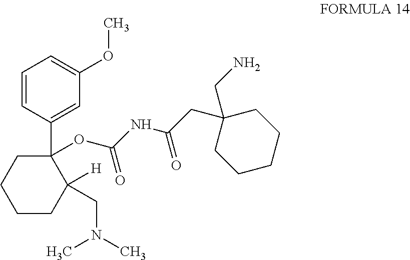

Another aspect of the invention relates to compounds of formula 15;

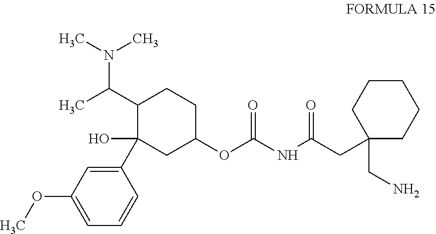

Another aspect of the invention relates to compounds of formula 16;

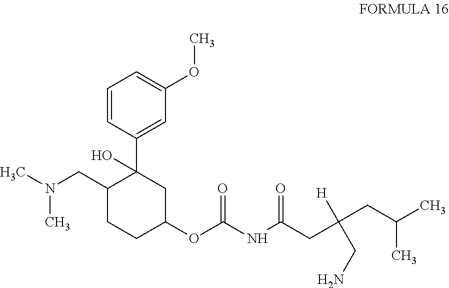

Another aspect of the invention relates to compounds of formula 17;

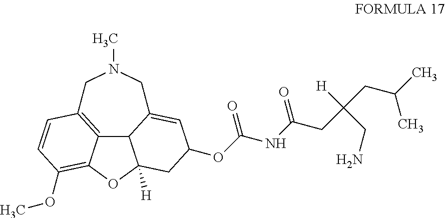

Another aspect of the invention relates to compounds of formula 18;

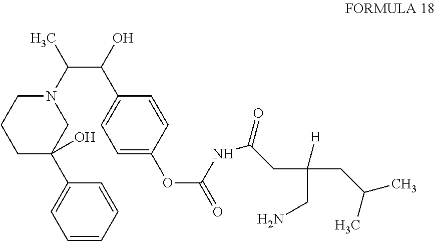

Another aspect of the invention relates to compounds of formula 19;

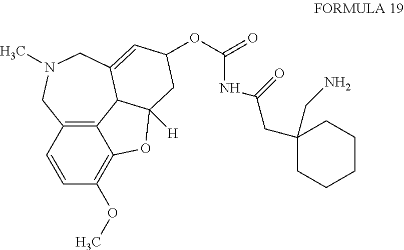

Another aspect of the invention relates to compounds of formula 20;

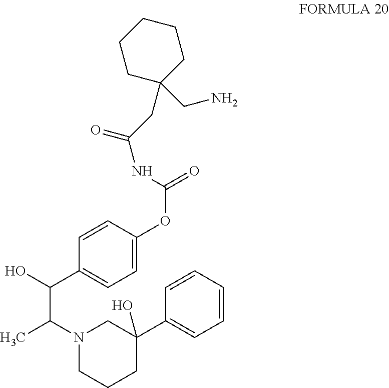

Another aspect of the invention relates to compounds of formula 21;

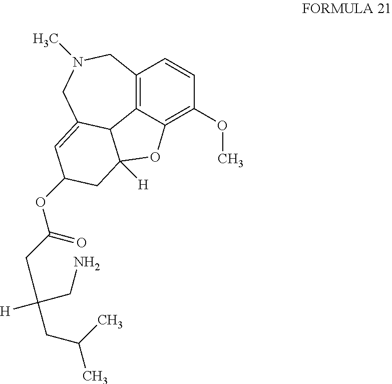

Another aspect of the invention relates to compounds of formula 22;

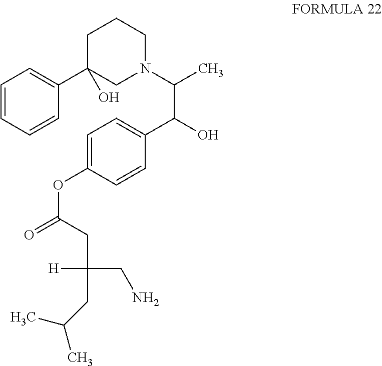

Another aspect of the invention relates to compounds of formula 23;

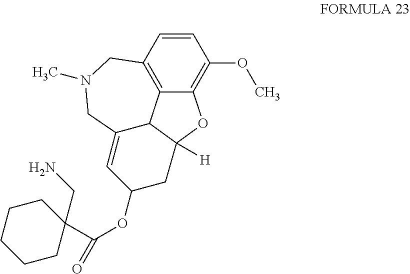

Another aspect of the invention relates to compounds of formula 24;

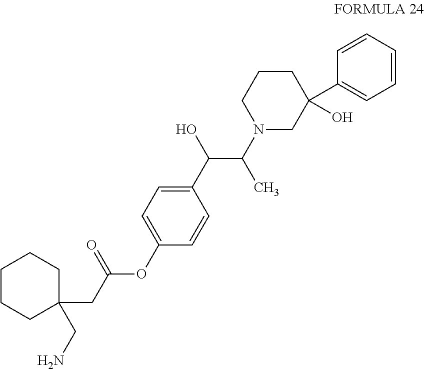

Another aspect of the invention relates to compounds of formula 25;

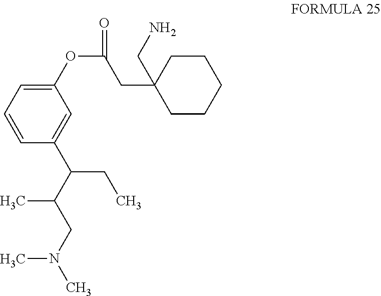

Another aspect of the invention relates to compounds of formula 26;

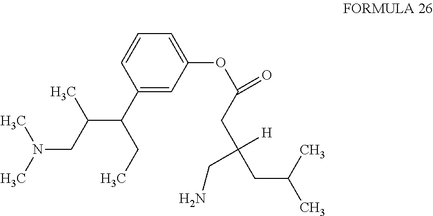

Another aspect of the invention relates to compounds of formula 27;

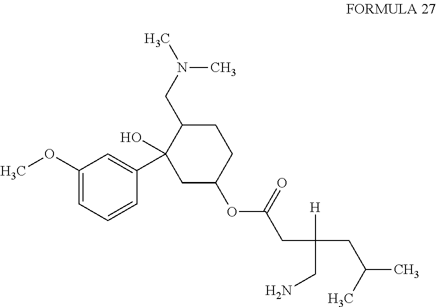

Another aspect of the invention relates to compounds of formula 28;

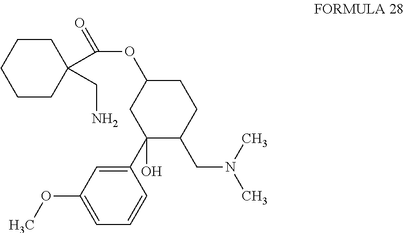

Another aspect of the invention relates to compounds of formula 29;

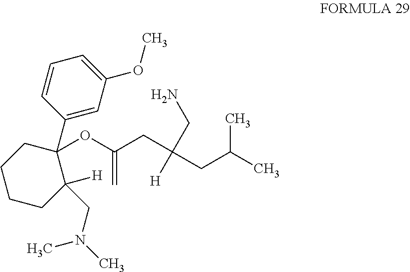

Another aspect of the invention relates to compounds of formula 30;

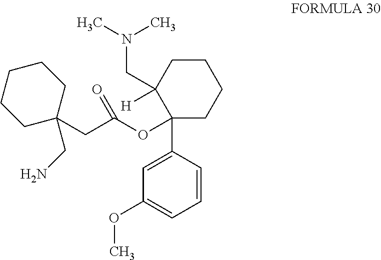

Another aspect of the invention relates to compounds of formula 31;

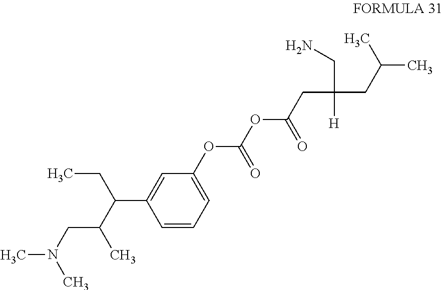

Another aspect of the invention relates to compounds of formula 32;

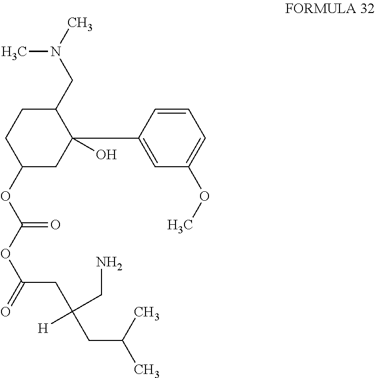

Another aspect of the invention relates to compounds of formula 33;

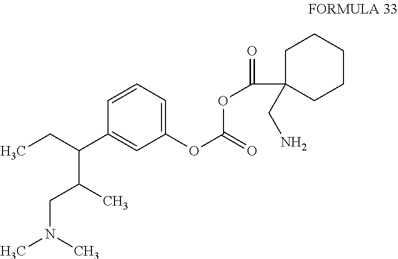

Another aspect of the invention relates to compounds of formula 34;

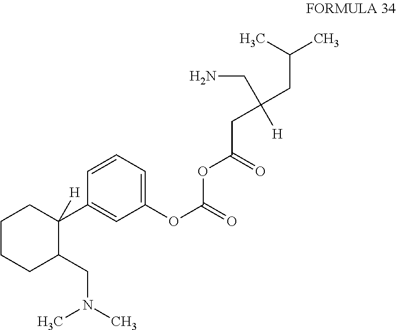

Another aspect of the invention relates to compounds of formula 35;

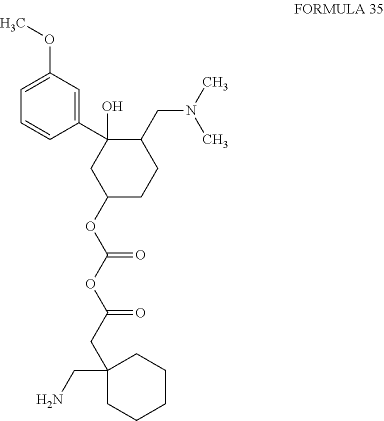

Another aspect of the invention relates to compounds of formula 36;

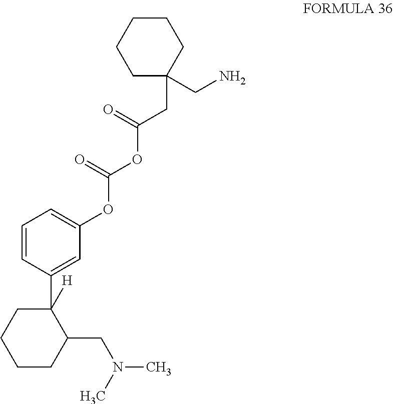

Another aspect of the invention relates to compounds of formula 37;

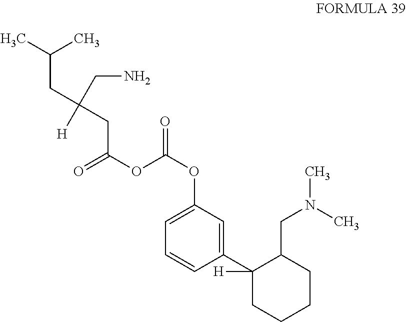

Another aspect of the invention relates to compounds of formula 38;

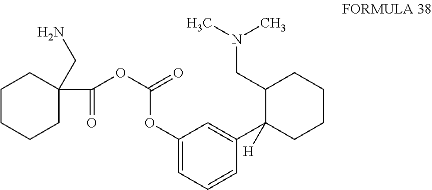

Another aspect of the invention relates to compounds of formula 39;

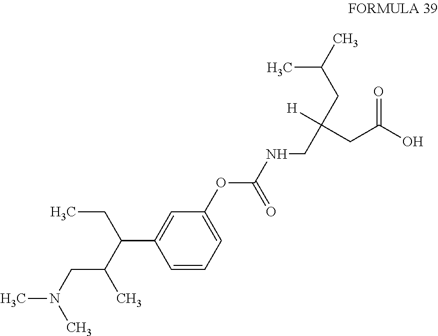

Another aspect of the invention relates to compounds of formula 40;

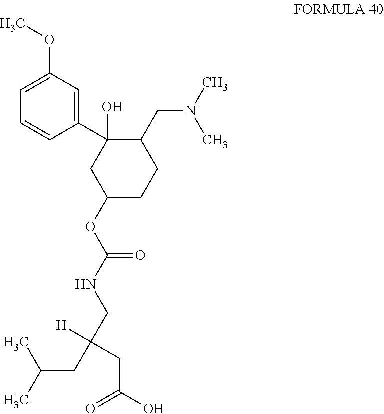

The following Examples are provided for illustrations and are in no way limit the scope of this invention.

### EXAMPLE I

### Preparation of Compound of Formula 1

The starting materials (3S)-3-(aminomethyl)-5-methylhexanoic acid (FORMULA I) and 3,5-dimethyltricyclo[3.3.1.13,7]decan-1-amine (FORMULA VIII) purified by standard recrystalization with Isopropyl Alcohol and Ethanol respectively. ((3S)-3-(aminomethyl)-5-methylhexanoic acid (FORMULA I) as converted into its acid chloride (3S)-3-(aminomethyl)-5-methylhexanoyl chloride either using Thionyl Chloride or Phosphorus Trichloride using standard procedures. The purified (3S)-3-(aminomethyl)-5-methylhexanoyl chloride was combined with 3,5-dimethyltricyclo[3.3.1.13,7]decan-1-amine (FORMULA VIII). For Example; To 500 ml. of dichloromethane taken in a 1000 ml. round bottom flask under nitrogen purge, was added 100.0 gm. of 3,5-dimethyltricyclo[3.3.1.13,7]decan-1-amine. While cooling the flask in an ice water bath, 65.4 gm. of (3S)-3-(aminomethyl)-5-methylhexanoyl chloride were added at a rate sufficient to maintain the temperature of the reaction mixture at between about 20′ C and 25′ C. The ice bath was removed and the mixture stirred for one hour. Then added to the mixture while still stirring, 6.1 gm. of thionyl chloride. The mixture continued to be stirred for one hour. Then the solvent and other volatiles were vacuum distilled, leaving a heavy amber-colored oil. To it were added 287 ml. of ethyl acetate and the mixture was stirred for two hours resulting in a fine white precipitate. These white solid particles were collected under vacuum on a Buchner funnel and washed thoroughly with ethyl acetate. Upon drying at 80′ C., a yield of 100.7 gm. Compound XX was recovered having analytical purity. The Molecular weight: 320.51, The part I was exhibited ¹HNMR (CDCl₃, 400 MHZ): δ0.833 (6H, singlet), 1.156 (2H, quartet), 1.328 (4H, quartet), 1.683 (4H, quartet), 1.869 (2H, broad signal), 2.179 (1H, broad signal), 8.28(3H, broad signal) and the part II exhibited the following; 52.95 (IH, dd, J=12.84 Hz and 3.54 Hz), 2.82 (IH, dd, J=12.82 Hz and 7.94 Hz), 2.44 (IH, dd, J=15.73 Hz and 3.37 Hz), 2.25 (IH, dd, J=15.70 Hz and 8.76 Hz), 2.06(1H, m), 1.69 (IH, m), 1.23 (2H, m), 0.92 (6H, t, J=6.42 Hz).

Mass Spectra; MS (Q-T of micro, ESI⁺): 179 (M⁺ 320.28).

Element Analysis (C₂₀H₃₆N₂O), (Actual results) Calculated value %: C(74.95%) 74.93%, H(11.32%)11.33%, N(8.74%) 8.73% O(4.99%) 4.99%

### EXAMPLE 2

Preparation of Compound of Formula 31:

3-[(2R,3R)-1-(dimethylamino)-2-methylpentan-3-yl]phenol (FORMULA IX) was treated with para nitrophenyl chloroformate in triethyl amine/Dry Chloroform at 0″ C to convert into its para nitro phenoxy carbonate ester according to Scheme IV. For Example 100 mg 3-[(2R,3R)-1-(dimethylamino)-2-methylpentan-3-yl]phenol was dissolved in 5 ML of dry Chloriform under inert atmosphere in a round-bottom flask. The solution was cooled down to 0′ C and 0.05 ml of triethyl amine was added very slowly and the reaction mixture was allowed to stir for 5 minutes. Para-nitrophenyl chloroformate 50 mg was dissolved in 10 mL of dry chloroform and was added to the 3-[(2R,3R)-1-(dimethylamino)-2-methylpentan-3-yl]phenol reaction mixture slowly and the reaction mixture was then allowed to warm to room temperature. The completion of the reaction was determined by thin layer chromatography. The resulting solution was dried by removing the solvents under vacuum to obtain an oily solid material which is dissolved and dried repeatedly to remove the unwanted by products. The Para nitrophenoxy carbonate ester of 3-[(2R,3R)-1-(dimethylamino)-2-methylpentan-3-yl]phenol (Yield 22%). The resulting Para nitrophenoxy carbonate ester of 3-[(2R,3R)-1-(dimethylamino)-2-methylpentan-3-yl]phenol in turn treated with 3S-3-(aminomethyl)-5-methylhexanoic acid (FORMULA I) in triethyl amine and THF solvent at 0′ C. For Example: 100 mg of 3S-3-(aminomethyl)-5-methylhexanoic acid (FORMULA I) was dissolved in 5 ml tetrahydrofuran solvent under inert conditions (Nitrogen gas) and 0.05 ml of triethyl amine was added gradually over a period of time and cooled to 0′ C. Separately 100 mg of para nitrophenoxy carbonate ester of 3-[(2R,3R)-1-(dimethylamino)-2-methylpentan-3-yl]phenol was dissolved in 5 ml of dry Tetrahydrofuran solvent by constant stirring. The resulting reaction mixture was cooled to 0′ C. The two reaction mixtures were gradually mixed by constant stirring at 0′ C under inert conditions and the reaction mixture was allowed to gradually warm up room temperature. The compound of Formula 31 was extracted by standard work up and purified by chromatography (Yield 15%).

### Clinical Experiments

The novel compounds of this invention are of significant therapeutic benefit and it is demonstrated in a clinical study involving tail-flick test on rats was performed according to the following procedure:

Therapeutic Solutions: The compound 2 90 mg was dissolved in 30 ml of saline solution and the injection volume was calculated as 1 ml/kg to achieve 3 mg/kg of compound of Formula 31. The placebo solution was identical except it is devoid of any drug.

Tail-Flick Test: The experimentation was started by selecting ten at least 90-100 days old and were all weighed to determine the volume of experimental and placebo solution. Each rat, was habituated for three days to handling and the tail flick procedure and without heat exposure. In the administration, each animal was only used for one day of experiments, and given only one dose of drug, or placebo solution. The technique of D'Amour and Smith (1941) relies on latency to flick of the tail from onset of a focused beam of intense light. This method, employed for decades, has now been augmented by the ability to monitor temperature at the site of exposure. Columbus Instruments model TF-2 model was warmed, on the day of the experiment, for at least 30 minutes. The intensity of the lamp was adjusted so that baseline tail-flick latency for rats is equal to approximately 2.0 seconds. The intensity was set to 40% as this was determined to be the ideal intensity from the intensity response curve. The apparatus should be programmed to use a cut-off point of 10 seconds to prevent tissue damage to the rats in the case that the tail does not flick. The experimental rat was placed in mitten and its tail was blackened for suitable length. The tail was placed in the groove and heat exposure was started. The lamp of the apparatus shuts down automatically whenever the tail flicks from the heat source. The baseline value was determined for each animal prior to injection. The TFL was measured approximately every hour. The semilog means were calculated.

### Results

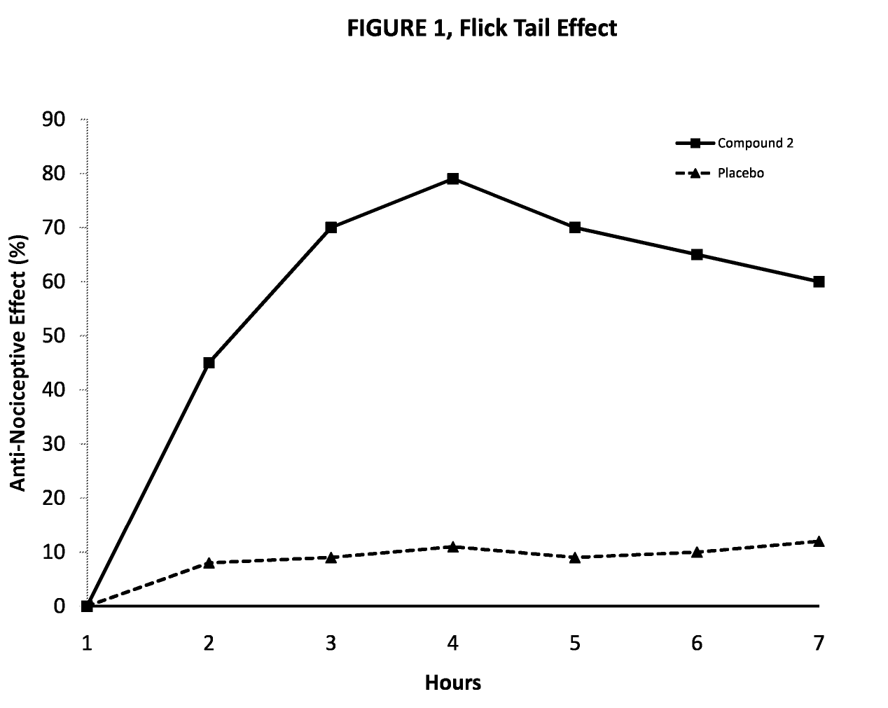

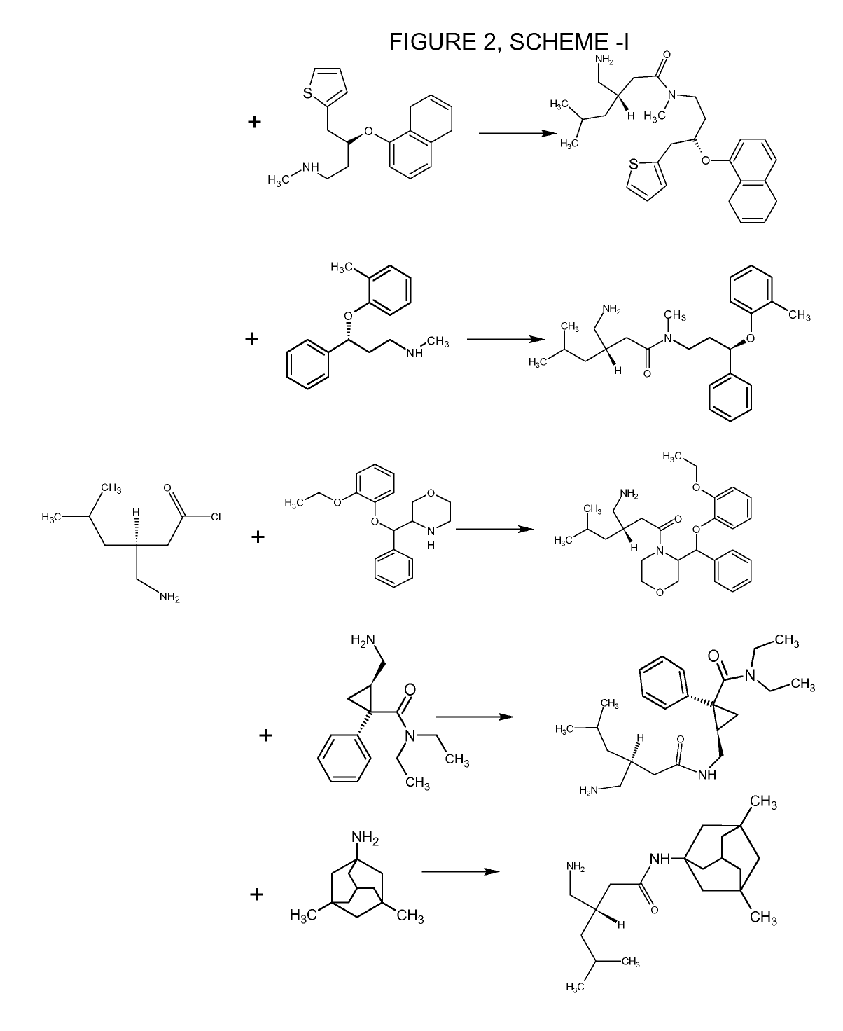

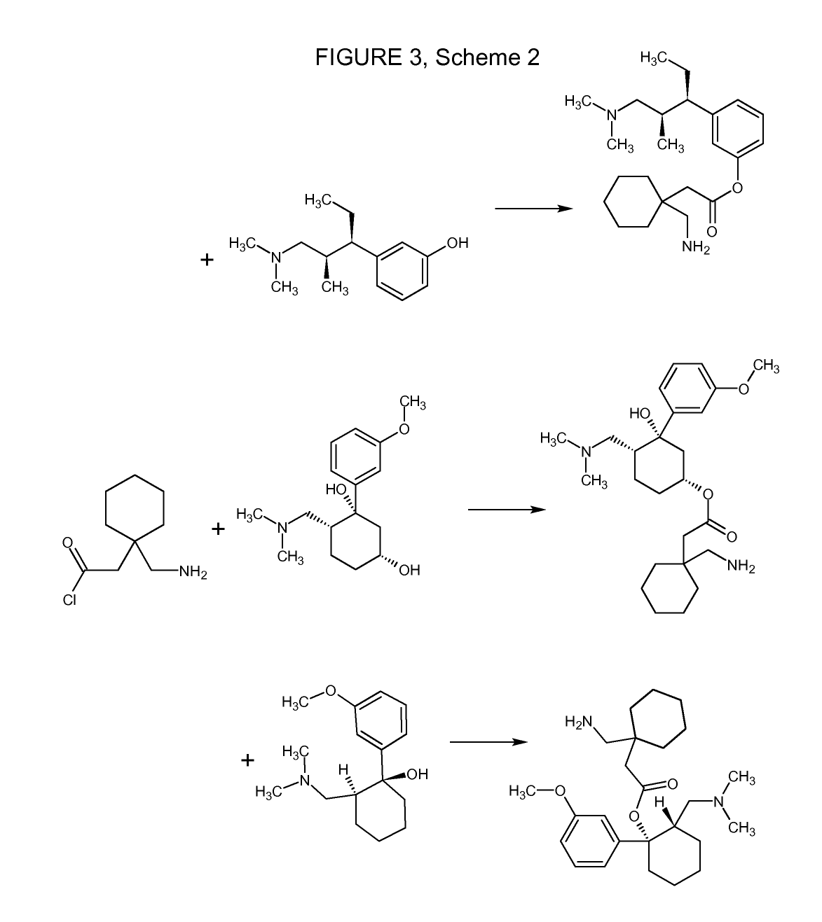

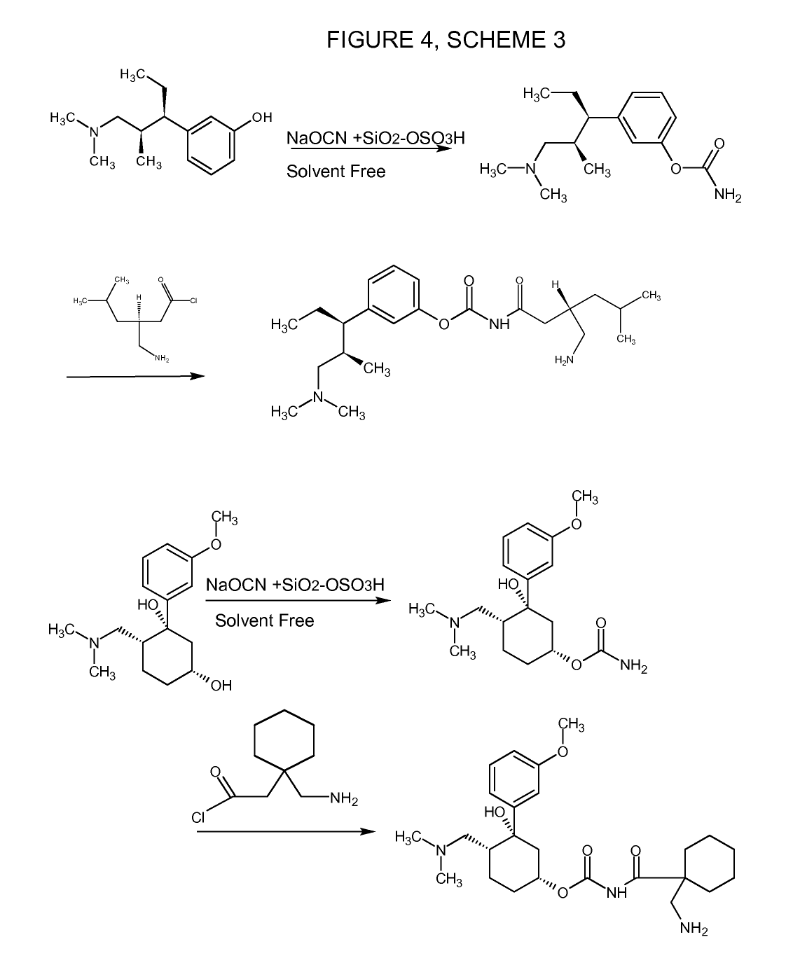

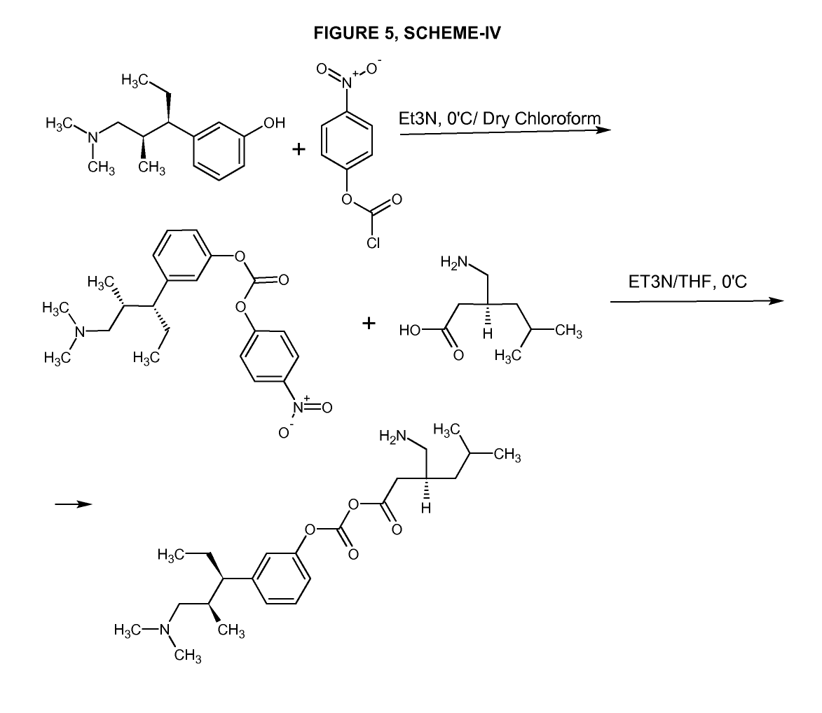

The invention discloses a number of novel therapeutic compounds that are useful for treating disorder in a mammal as demonstrated in FIG. 1.

1. A compound according to claim 43, which has the formula 1:

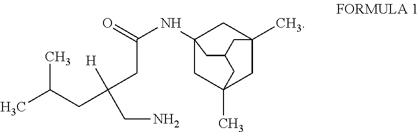

2. A compound according to claim 43, which has the formula 2:

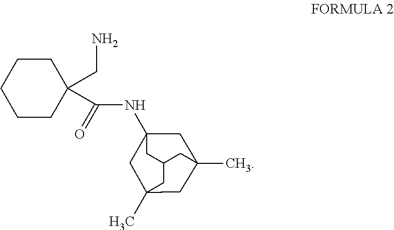

3. A compound according to claim 43, which has the formula 3;

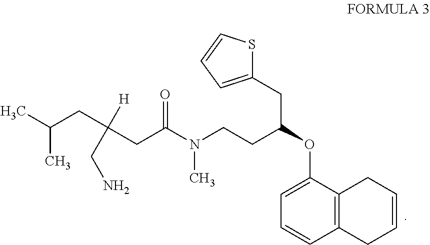

4. A compound according to claim 43, which has the formula 4;

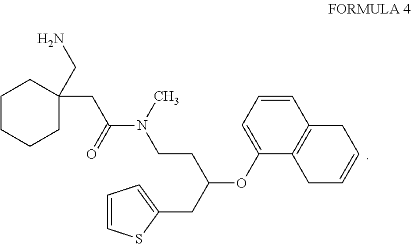

5. A compound according to claim 43, which has the formula 5;

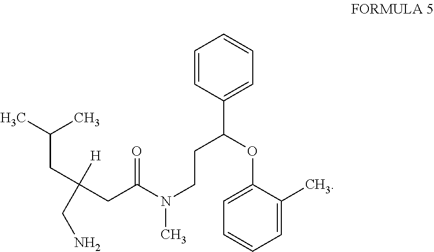

6. A compound according to claim 43, which has the formula 6;

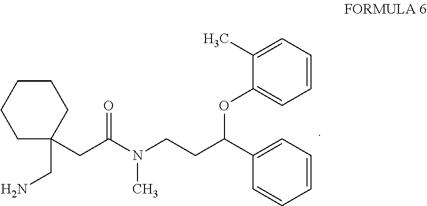

7. A compound according to claim 43, which has the formula 7;

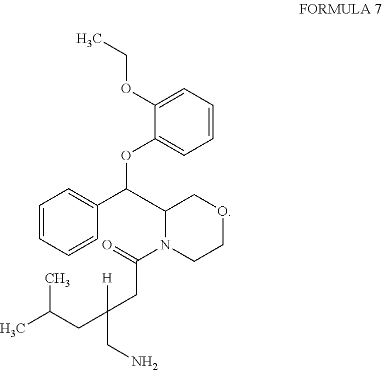

8. A compound according to claim 43, which has the formula 8;

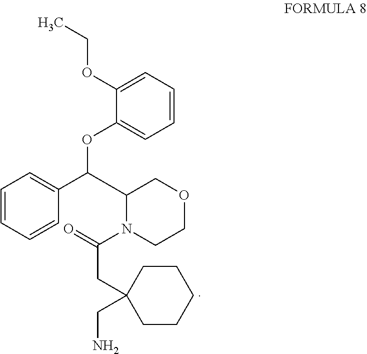

9. A compound according to claim 43, which has the formula 9;

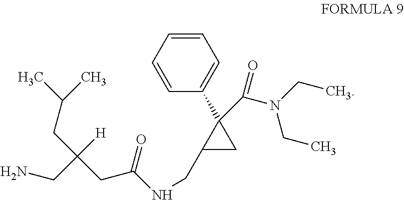

10. A compound according to claim 43, which has the formula 10;

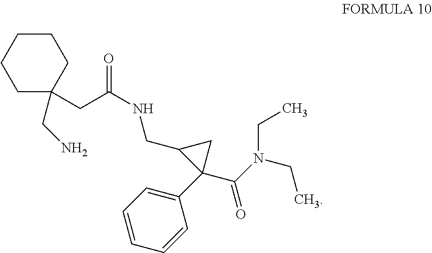

11. A compound according to claim 43, which has the formula 11;

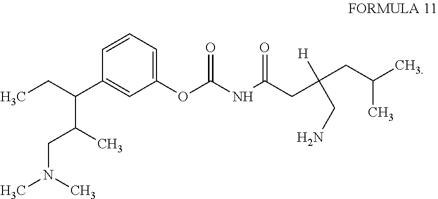

12. A compound according to claim 43, which has the formula 12;

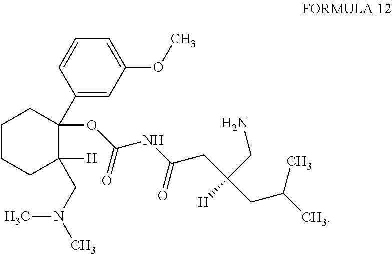

13. A compound according to claim 43, which has the formula 13;

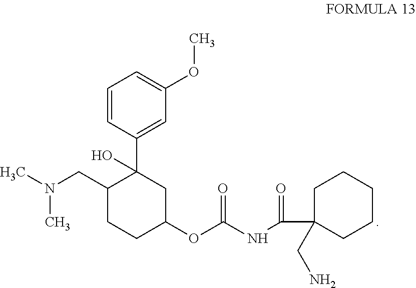

14. A compound according to claim 43, which has the formula 14;

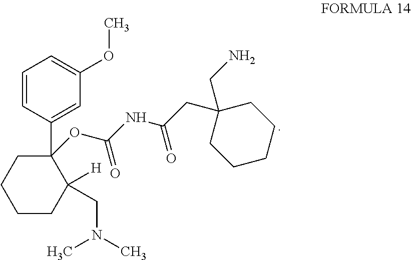

15. A compound formula 15;

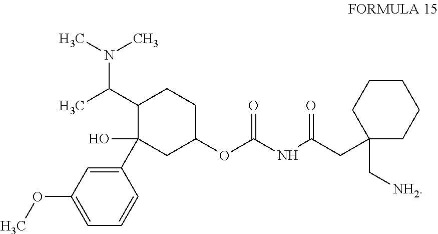

16. A compound according to claim 43, which has the formula 16;

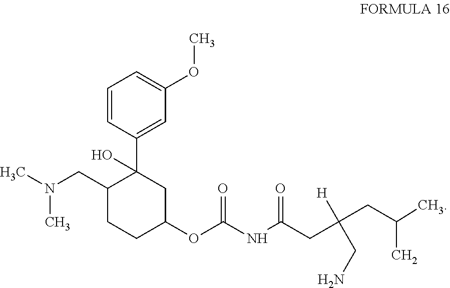

17. A compound according to claim 43, which has the formula 17;

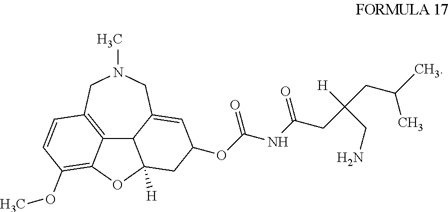

18. A compound according to claim 43, which has the formula 18;

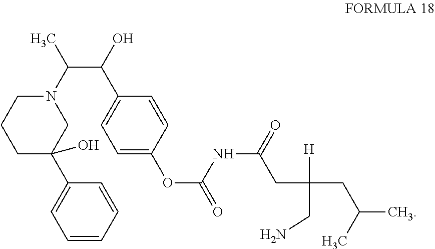

19. A compound according to claim 43, which has the formula 19;

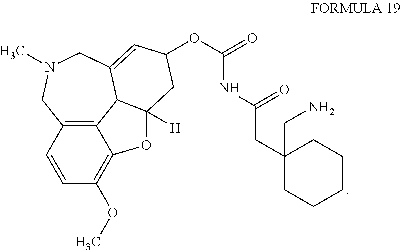

20. A compound compounds according to claim 43, which has the formula 20;

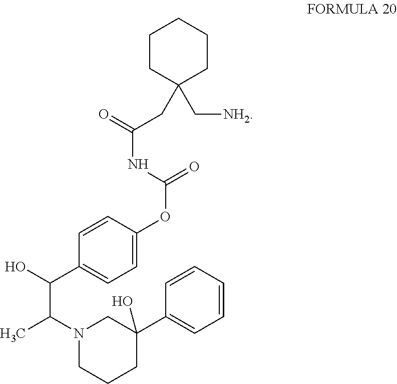

21. A compound according to claim 43, which has the formula 21;

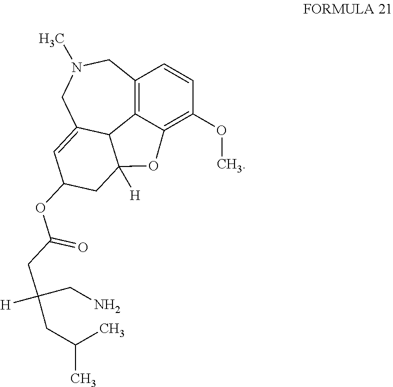

22. A compound according to claim 43, which has the formula 22;

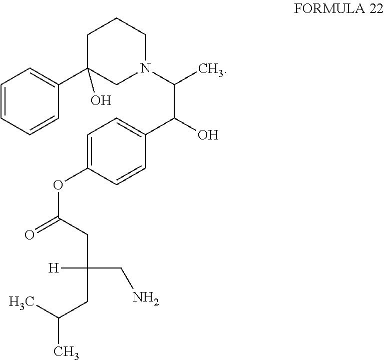

23. A compound according to claim 43, which has the formula 23;

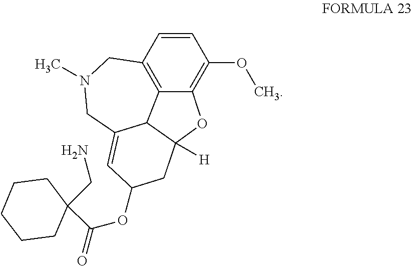

24. A compound according to claim 43, which has the formula 24;

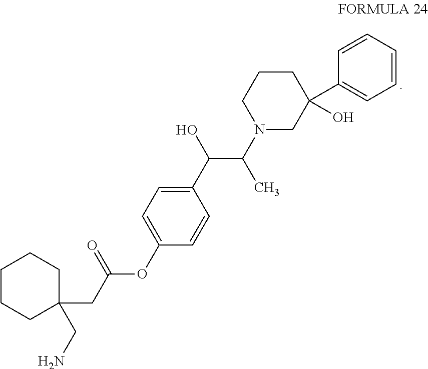

25. A compound according to claim 43, which has the formula 25;

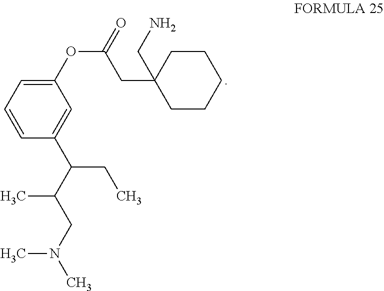

26. A compound according to claim 43, which has the formula 26;

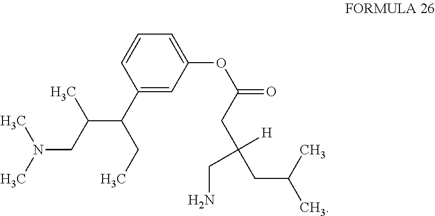

27. A compound according to claim 43, which has the formula 27;

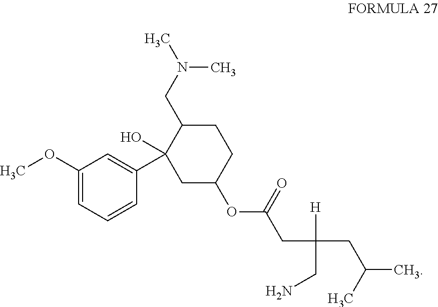

28. A compound according to claim 43, which has the formula 28;

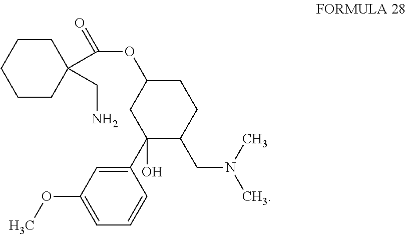

29. A compound according to claim 43, which has the formula 29;

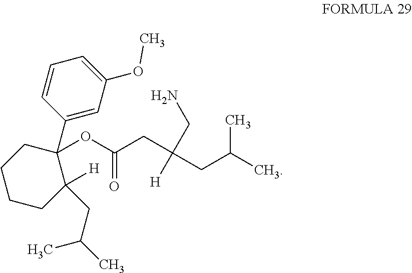

30. A compound according to claim 43, which has the formula 30;

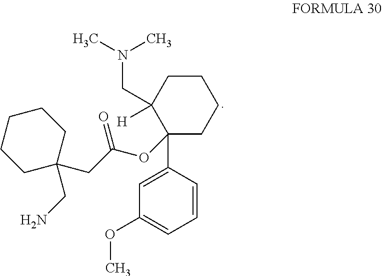

31. A compound according to claim 43, which has the formula 31;

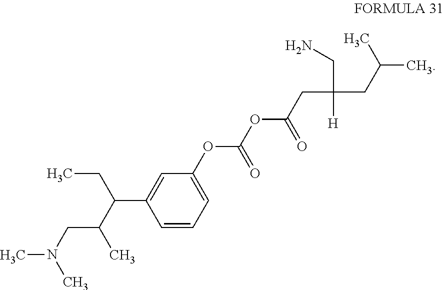

32. A compound according to claim 43, which has the formula 32;

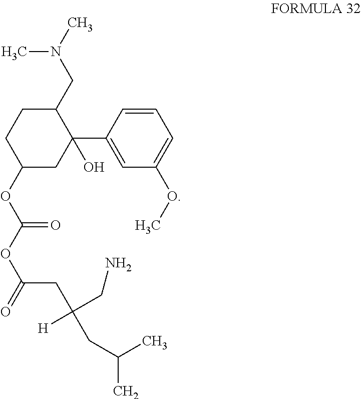

33. A compound according to claim 43, which has the formula 33;

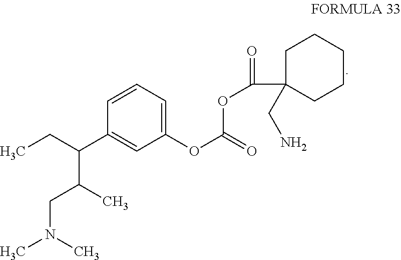

34. A compound according to claim 43, which has the formula 34;

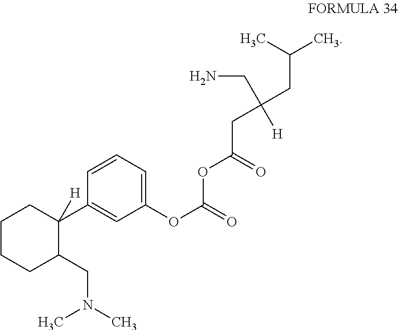

35. A compound according to claim 43, which has the formula 35;

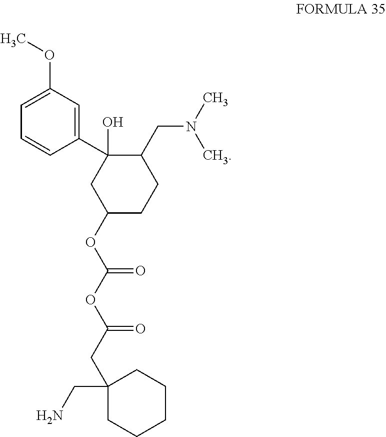

36. A compound according to claim 43, which has the formula 36;

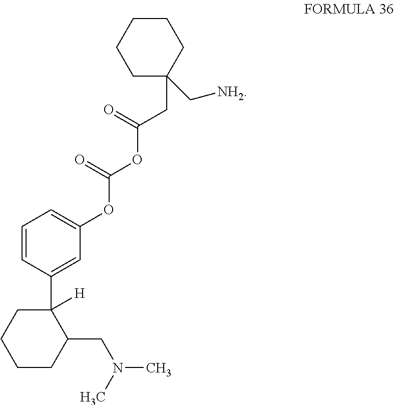

37. A compound according to claim 43, which has the formula 37;

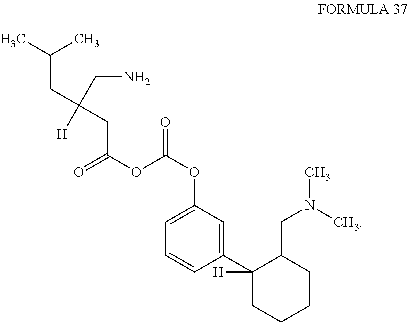

38. A compound according to claim 43, which has the formula 38;

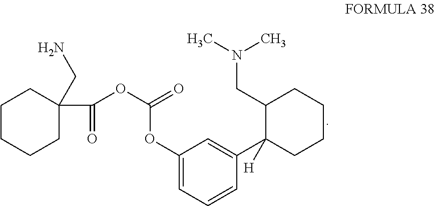

39. A compound according to claim 43, which has the formula 39;

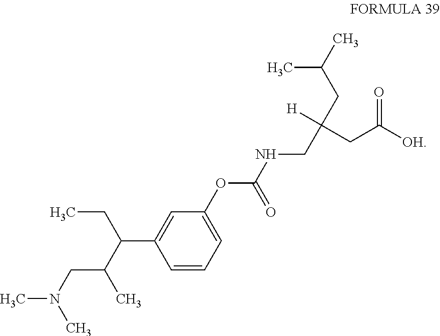

40. A compound according to claim 43, which has the formula 40;

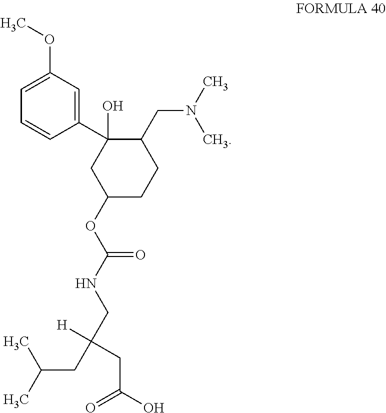

41. A method of treating a disorder in a mammal comprising administering to said mammal an effective amount of a compound according to claim 43.

42. A pharmaceutical composition comprising a therapeutically effective amount of a compound according to claim 43.

43. A compound selected from the group consisting of: 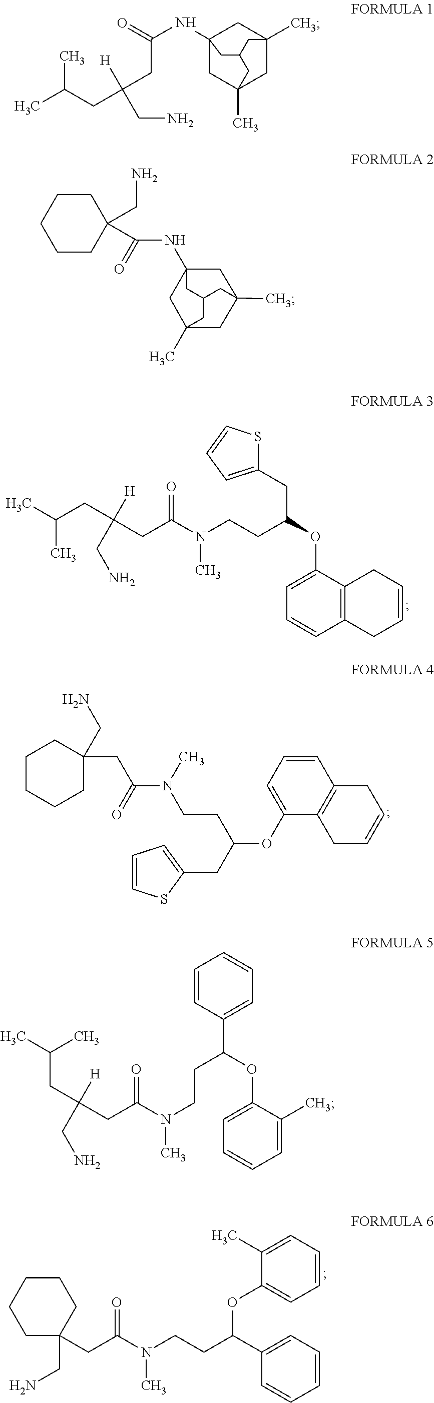 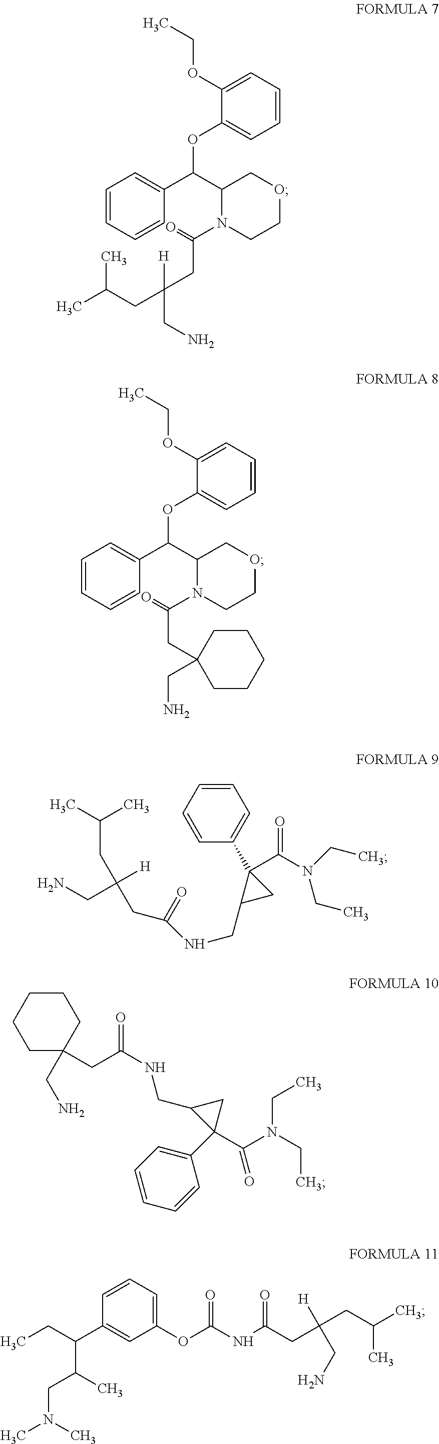 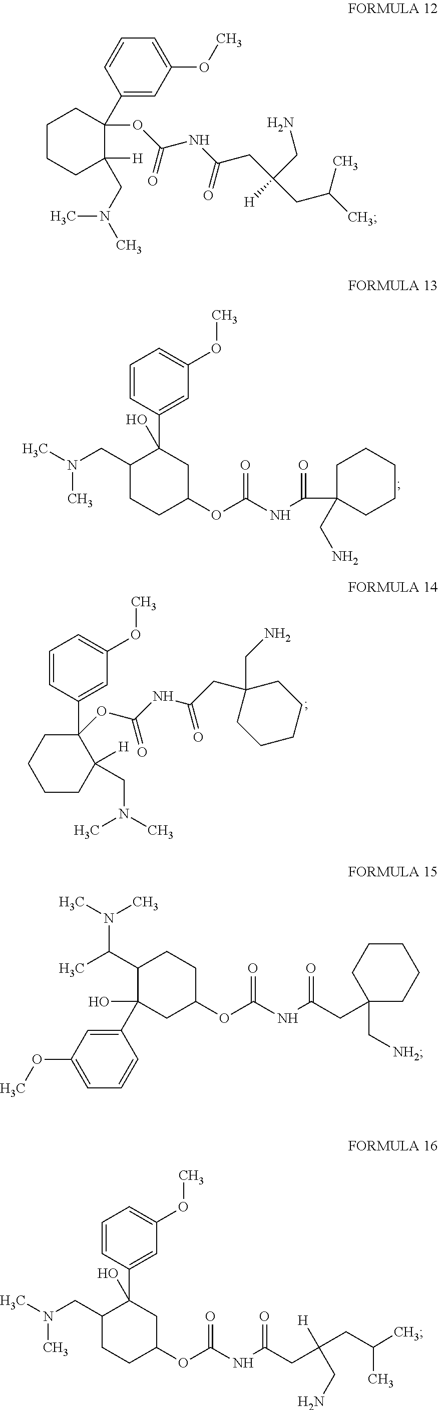 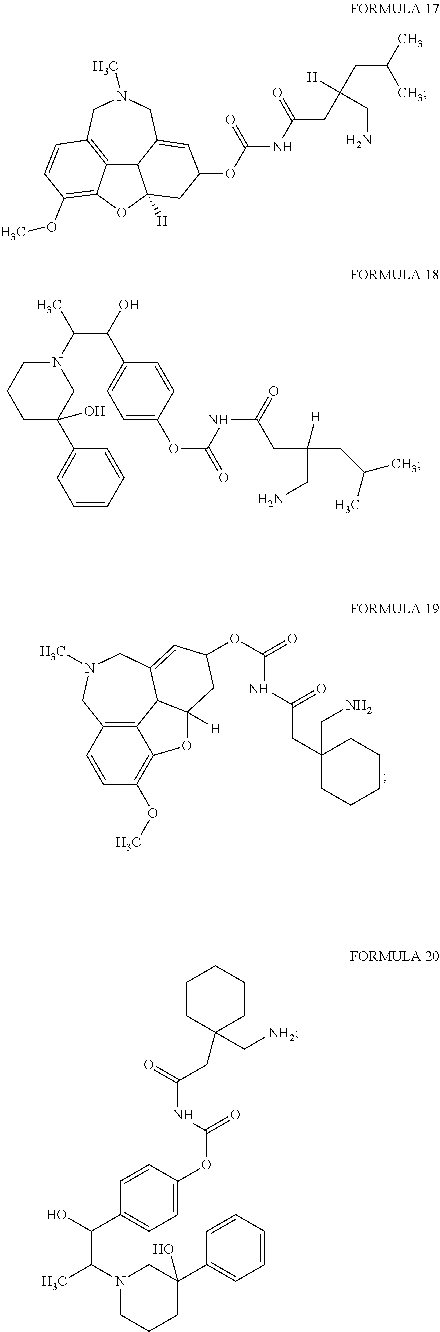 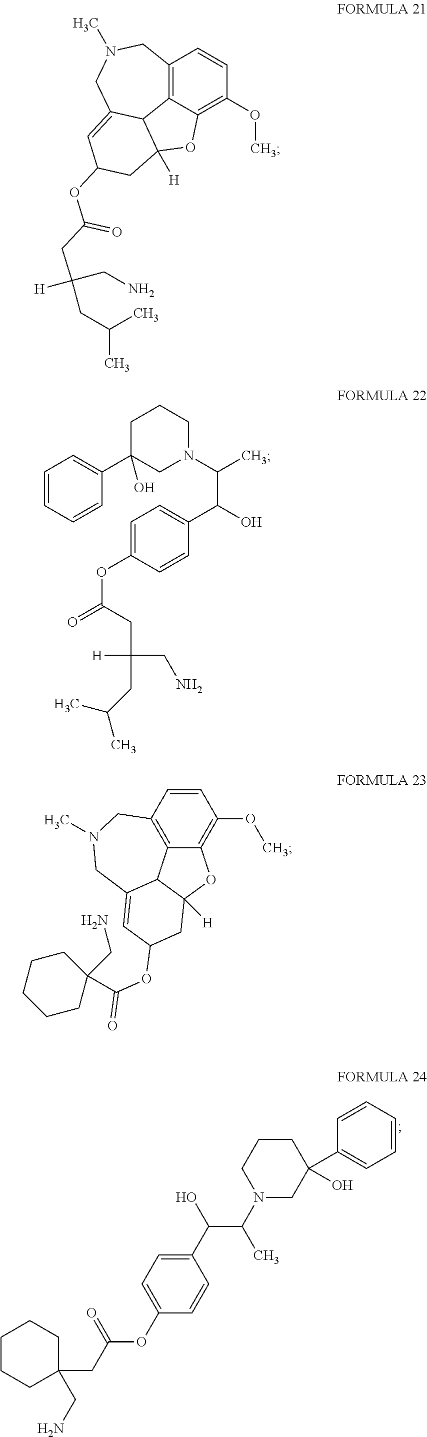 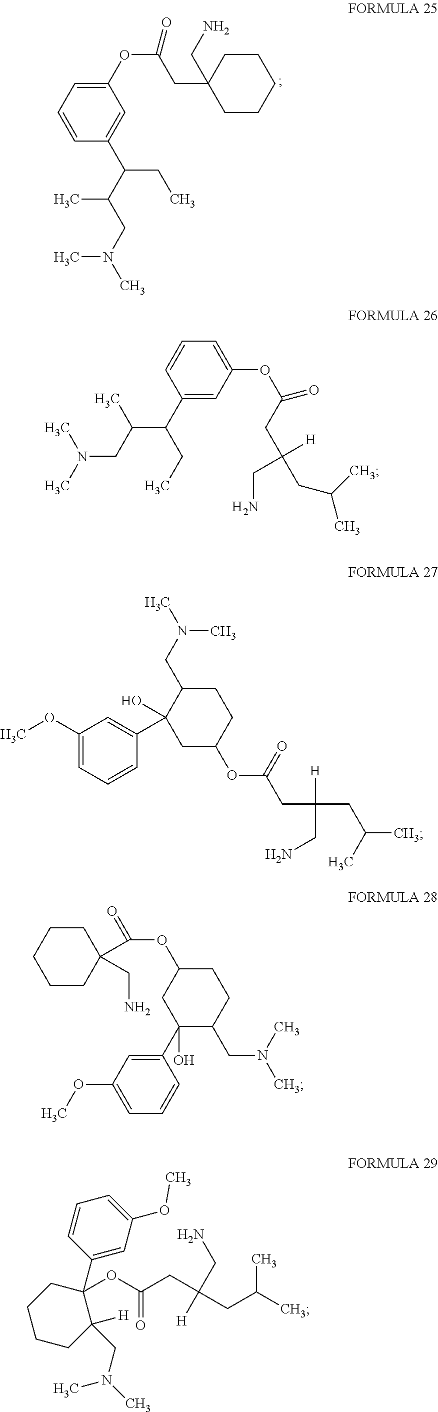 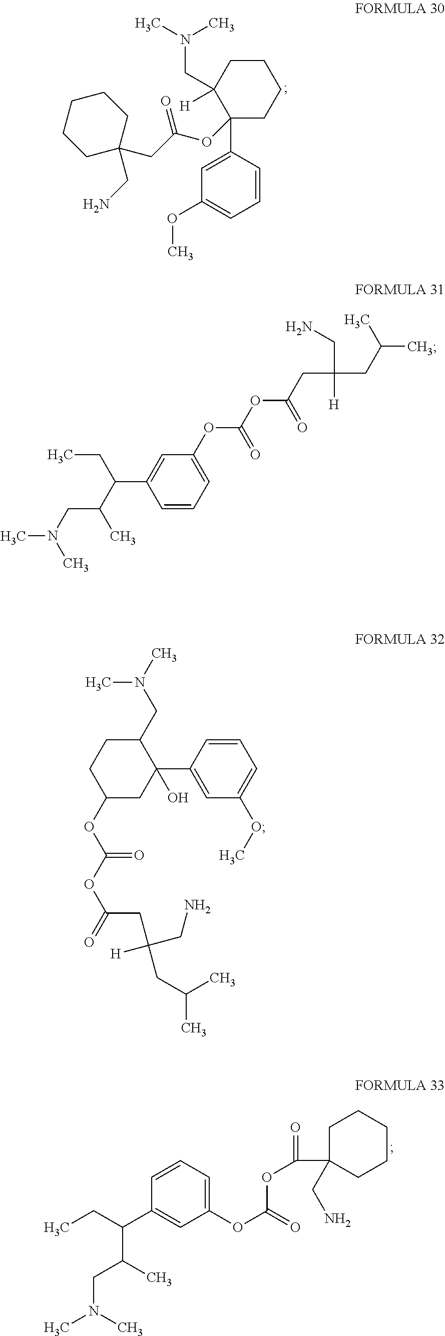 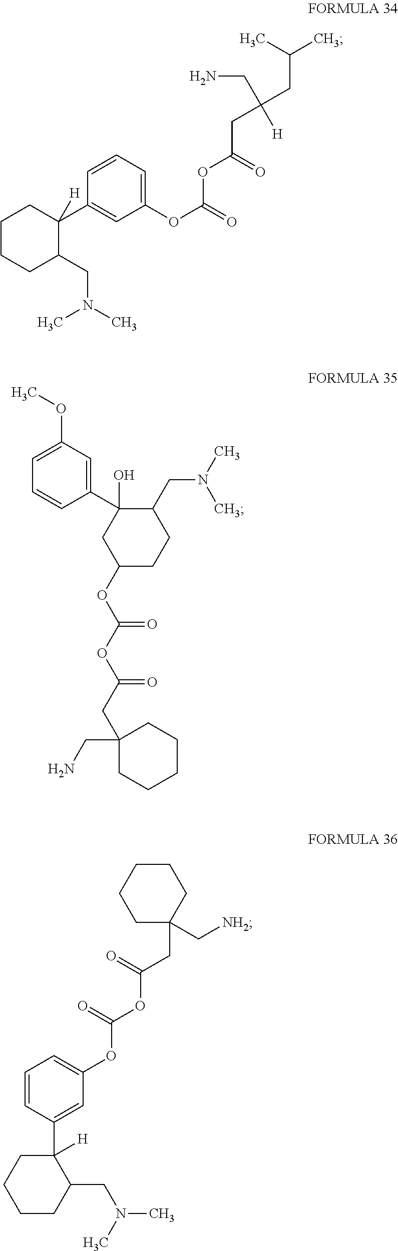 [chemical structure]
